SEQUENCE LISTING (1 sequence)

SEQ ID NO: 1 - protein, 377 aa, Homo sapiens
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

(19) United States
(12) Patent Application Publication
(10) Pub. No.: US 2026/0199348 A1
(43) Pub. Date: Jul. 16, 2026

(54) TREATMENT OF MULTISYSTEMIC SMOOTH MUSCLE DYSFUNCTION SYNDROME

(71) Applicants: QRGENETICS LTD., Tel Aviv (IL); The General Hospital Corporation, Boston, MA (US)

(72) Inventors: Shane WALD-ALTMAN, Ramat HaSharon (IL); Patricia L. MUSOLINO, Belmont, MA (US); Vijaisrinivasan KRISHNAN, Revere, MA (US)

(21) Appl. No.: 18/965,466

(22) Filed: Dec. 2, 2024

Related U.S. Application Data
(60) Provisional application No. 63/604,788, filed on Nov. 30, 2023.

(51) Int. Cl. A61K 31/519; A61K 45/06; A61P 21/00; C12Q 1/6883

(52) U.S. Cl. CPC A61K 31/519; A61K 45/06; A61P 21/00; C12Q 1/6883; C12Q 2600/156

(57) ABSTRACT

A method of pharmacologically treating a subject being an MSMDS patient or a subject having or suspected of having a substitution in an actin alpha (ACTA2) locus, comprising administering a therapeutically effective amount of Sapropterin to a subject, contingent upon a presence of a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S).

### CROSS-REFERENCE TO RELATED APPLICATIONS

This application claims the benefit of priority of U.S. Provisional Patent Application No. 63/604,788 filed on Nov. 30, 2023, incorporated by reference as if fully set forth herein in their entirety.

### REFERENCE TO A SEQUENCE LISTING

The application contains a Sequence Listing which has been submitted electronically in XML format and is hereby incorporated by reference in its entirety. Said XML copy, created on Dec. 2, 2024, is named QRG0019US01 Seq listing.xml and is 2 Kilobytes in size. The information contained in the Sequence Listing is incorporated by reference herein in its entirety.

### TECHNICAL FIELD

Described herein are compositions and methods of treatment of Multisystemic Smooth Muscle Dysfunction Syndrome (MSMDS) using Sapropterin.

### BACKGROUND

Genetic vasculopathies cause up to 10% of childhood strokes (10-20/100,000, over 8,000 cases per year). The majority affect pericytes and smooth muscle cell (SMC) function and are caused by missense mutations. One of the most prototypical and severe SMC vasculopathies is caused by a pathogenic missense mutation in the actin alpha 2 smooth muscle gene, ACTA2, at arginine 179 (most commonly replaced by histidine and referred herein ACTA2 R179H) which leads to multisystemic smooth muscle dysfunction syndrome, herein referred to as “MSMDS”. Multisystemic smooth muscle dysfunction syndrome is characterized by systemic smooth muscle cell dysfunction, hypotension, aortic aneurysms and a devastating cerebrovascular disease that leads to death in the first 3 decades of life.

MSMDS patients experience a wide array of clinical signs and symptoms, including congenital mydriasis, patent ductus arteriosus (PDA), pulmonary artery hypertension, aortic and other arterial aneurysms, moyamoya-like cerebrovascular disease, intestinal hypoperistalsis and malrotation, and hypotonic bladder (see Regalado, E. S., et al.). Risk of mortality is high during childhood, and currently, there is no approved treatment for MSMDS.

### SUMMARY

This Summary is provided to introduce a selection of concepts in a simplified form that are further described below in the Detailed Description. Other features, details, utilities, and advantages of the claimed subject matter will be apparent from the following written Detailed Description including those aspects illustrated in the accompanying drawings and defined in the appended claims.

Sapropterin, a synthetic form of tetrahydrobiopterin, was first marketed as Kuvan® and was approved by the FDA for the treatment of phenylketonuria, the most common metabolic disorder affecting children and adults. However, its potential therapeutic effects on MSMDS have not been explored previously.

The inventors of the present invention have discovered a pharmacological approach using Sapropterin, for the treatment of Multisystemic Smooth Muscle Dysfunction (MSMDS). Based on their research, Sapropterin was found to compensate for actin dysfunction in human patient-derived fibroblasts, as well as treating or preventing the development of disease in a mouse model of Multisystemic Smooth Muscle Dysfunction Syndrome (MSMDS).

Thus, the present invention relates to methods of treatment, and combination compositions which remedy the long-felt need for improved treatment for patients suffering from Multisystem smooth muscle dysfunction syndrome (MSMDS). The therapeutic agents and methods provided are based, at least in part, on the discovery that Sapropterin is specifically capable of improving or compensating for dysfunctional activity in the actin alpha 2 smooth muscle protein, in the context of Multisystem smooth muscle dysfunction syndrome (MSMDS).

In a first aspect, there is provided a method of treating an MSMDS patient comprising administering a therapeutically effective amount of Sapropterin.

In another aspect, there is provided a method of treating a subject having or suspected of having a substitution in an actin alpha (ACTA2) locus, comprising administering a therapeutically effective amount of Sapropterin to the patient or subject, contingent upon the presence of a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S). For example, Sapropterin compositions may be provided for use in treating MSMDS in a subject who has a genetic substitution in the actin alpha (ACTA2) locus wherein the genetic substitution results in an amino acid change, at position 179, of arginine for histidine (R179H).

In some embodiments, the administering a therapeutically effective amount of Sapropterin to the subject, may be systemically administered, by oral or intraperitoneal route.

In another aspect, treating a subject may be by using Sapropterin as pharmaco-therapeutic to improve function of cells harboring the ACTA2 R179H pathogenic variant.

Consistent with some disclosed embodiments, a method of treating a subject in a risk category for developing MSMDS may further include: obtaining a biological sample from the subject, detecting in the biological sample a presence of a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S); and administering a therapeutically effective amount of sapropterin to the subject when the substitution is present. For example, the substitution in an actin alpha 2 (ACTA2) locus may be a genetic substitution in the actin alpha (ACTA2) locus wherein the genetic substitution results in an amino acid change, at position 179, of arginine for histidine (R179H).

Consistent with some disclosed embodiments, a subject may refer to a pregnant mother carrying a fetus having a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S).

In another aspect, there is provided a kit comprising components for detecting in a biological sample obtained from a subject, a presence of a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S). For example, the kit may include components for detecting, in a biological sample obtained from a subject, a presence of a substitution of arginine for histidine (R179H), at position 179.

### BRIEF DESCRIPTION OF THE FIGURES

The patent or application file contains a least one drawing/photograph executed in color. Copies of this patent or patent application publication with color drawing(s)/photograph(s) will be provided by the Office upon request and payment of the necessary fee.

Aspects of the present disclosure are best understood from the following detailed description when read with the accompanying figures. It is emphasized that, under standard practice in the industry, various features are not drawn to scale. The dimensions of the various features may be arbitrarily increased or reduced for clarity of discussion. In addition, the present disclosure may repeat reference numerals and/or letters in multiple examples.

The foregoing and other features and advantages of the present invention will be more fully understood from the following detailed description of illustrative embodiments taken in conjunction with the accompanying drawings in which:

The following figures collectively illustrate development and characterization of a mouse model of MSMDS.

FIG. 1 depicts an Acta2 R179H mouse model allele structure (Allele name Acta2^{R179Hfl}). Cre excision results in the replacement of normal coding sequence with a new exon 6 coding for Histidine substitution at position 179. Further details and characterization of the model is disclosed in P.C.T. Patent Application No. WO2024073715, filed Sep. 23, 2023, the disclosure of which are all incorporated herein by reference as if fully set forth herein in its entirety.

FIGS. 2A, 2B, and 2C depict general ACTA2 vasculopathy and Pathomechanism.

FIGS. 3A and 3B depict molecular dynamic simulation of ACTA2 chains. FIG. 3A depicts Acta2 native behavior in healthy state. The R179 (arrow) binds to 193 and 196 residues marked in Blue (Orange chain). The binding distance between one chain to another is less than 5A, and it stays like this throughout the simulation signifying that the bond is very strong, enabling the proper function of the actin fiber. FIG. 3B depicts the effect of the Acta2 R179 mutation. The 179H binds to 193 and 196 residues marked in Blue (Orange chain) and the gap is observed between the two chains (circle), affecting their binding and functionality of the actin fiber.

FIGS. 4A-4D collectively illustrate in vitro characterization of dermal fibroblasts from control and ACTA2R179H patients. FIG. 4A depicts control and R179H mutant fibroblasts (untreated and Sapropterin treated), paraformaldehyde fixed and stained with phalloidin (fibrillary acting or F-actin stain) and DNASE1 (representative of globular actin or G-actin stain). High resolution imaging mutant R179H fibroblasts showed aberrant cellular morphology with loss of G-actin and F-actin fluorescent intensity signal. Treatment with sapropterin (100 μm) show phenotype reversal with G- and F actin fluorescence intensity levels comparable to control. Quantification of reduced G- and F-actin fluorescent intensity levels in mutant R179H fibroblasts compared to control. Sapropterin treatment (100 μm) reverses phenotype by significantly increasing G- and F-actin intensity levels.

FIGS. 5A-5D illustrate High Magnification Images of G- and F actin expression in untreated (FIG. 5A) vs sapropterin treated (FIG. 5B) fibroblasts. Control fibroblasts show dense fibrillar actin. Untreated mutant fibroblasts (FIG. 5C) show F actin degradation with loss of F-actin strands. Treatment with Sapropterin (FIG. 5D) significantly increasing G- and F-actin intensity levels and restoration of G/F actin ratio.

FIGS. 6A-6B collectively illustrate western blot quantification of G- and F-actin fractions and G/F actin ratio determination. FIG. 6A depicts a western blot gel showing protein expression bands for both G- and F actin fractions for conditions: untreated wildtype fibroblasts, Sapropterin treated wildtype fibroblasts, untreated R179H fibroblasts and Sapropterin treated R179H fibroblasts. FIG. 6B depicts quantification diagram showing the G/F actin fragment ratio, revealing a significantly higher ratio in mutant ACTA2 R179H fibroblasts compared to control. Treatment of mutant ACTA2 R179H fibroblasts with sapropterin restores the G/F actin ratio. This quantification diagram illustrates the measured G/F actin fragment ratio, providing valuable insights into actin function differences between control and mutant fibroblasts and the restorative effects of sapropterin treatment.

FIG. 7 is the experiment timeline.

FIGS. 8A-8B collectively depict the results of physiological in vivo testing using survival curve and body weight in measurements control Acta2^{R179Hfl/+} and Myh11-Cre:Acta2^{R179Hfl/+} mutant mice. FIG. 8A depict survival curve graph illustrating the survival rates of Myh11-Cre:Acta2^{R179Hfl/+} mutant mice, both untreated and sapropterin-treated. Untreated Myh11-Cre:Acta2^{R179Hfl/+} mutants exhibit decreased survival, with mortality observed as early as 4 weeks. In contrast, sapropterin-treated Myh11-Cre:Acta2^{R179Hfl/+} mutant mice demonstrate improved survival rates comparable to control mice. FIG. 8B: The body weight comparison diagram demonstrates the body weight differences between Myh11-Cre:Acta2^{R179Hfl/+} mutant mice and control Acta2^{R179Hfl/+} mice. Myh11-Cre:Acta2^{R179Hfl/+}. Myh11-Cre:Acta2^{R179Hfl/+} mutant mice exhibit lower body weights compared to controls. However, sapropterin-treated Myh11-Cre:Acta2^{R179Hfl/+} mutant mice show body weights physiologically comparable to control mice, indicating the efficacy of the treatment.

FIGS. 9A-9B depict Behavioral Assessments in Untreated and Sapropterin-Treated MSMDS Mouse Models Using Rotarod and Open Field Test. FIG. 9A rotarod motor data graph illustrates the motor coordination performance of Myh11-Cre:Acta2R179Hfl/+ mutant mice, both untreated and sapropterin-treated. Untreated Myh11-Cre:Acta2^{R179Hfl/+} mutants exhibit decreased rotarod performance from week 4 to week 8 compared to control mice. In contrast, sapropterin-treated Myh11-Cre:Acta2^{R179Hfl/+} mutant mice display restored motor activity, comparable to control Acta2fl−/+ mice. FIG. 9B depicts the exploratory behavior of control and mutant mice, using the open field test, and the data graph demonstrates a significantly increased response in sapropterin-treated Myh11-Cre:Acta2^{R179Hfl/+} mutant mice, indicating restored performance.

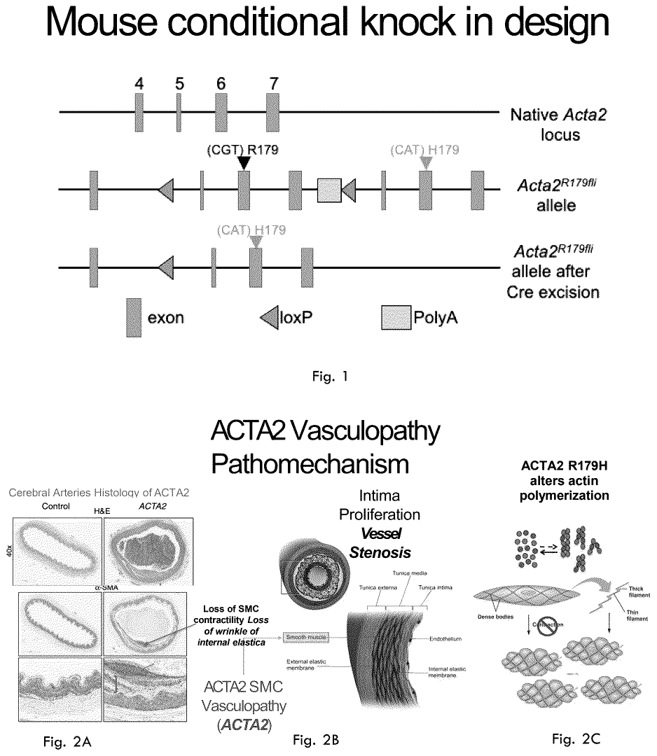

Advantages: The presented patent drawing offers the following advantages: Motor performance visualization: The rotarod motor data graph visually represents the differences in motor performance between untreated and sapropterin-treated Myh11-Cre:Acta2^{R179Hfl/+} mutant mice, providing clear evidence of the therapeutic effects of sapropterin on motor activity in the MSMDS mouse model. Locomotion and exploratory assessment: The open field test results depicted in the data graph highlight the significantly increased total and open field locomotion observed in sapropterin-treated Myh11-Cre:Acta2^{R179Hfl/+} mutant mice, indicating restored exploratory performance. Patent Claim: A patent claim is sought for the presented patent drawing, particularly the visual representation of sensory and motor behavioral assessments in untreated and sapropterin-treated MSMDS mouse models. The rotarod motor data graph and the open field test data graph provide evidence of the therapeutic efficacy of sapropterin in restoring motor performance and sensory function in the mouse model. Conclusion: The patent drawing presented herein visually demonstrates the innovative aspects of the invention, showcasing the effects of sapropterin treatment on sensory and motor behaviors in the MSMDS mouse model. The inclusion of the rotarod motor data graph and the open field test data graph strengthens the case for sapropterin as an effective treatment for MSMDS, supporting its potential as a therapeutic agent.

FIGS. 10A and 10B collectively show Systemic Blood Pressure Measurements in Untreated, FIG. 10A, and Sapropterin-Treated, FIG. 10B, MSMDS Mouse Model. The representative raw data trace depicts the low basal blood pressure observed in Myh11-Cre:Acta2^{R179Hfl/+} mutant mice. (FIG. 10B) The representative data demonstrates the increased blood pressure values observed in Sapropterin-treated Myh11-Cre:Acta2^{R179Hfl/+} mutant mice. In FIG. 10C, ta data comparison graph illustrates a significant reduction in blood pressure in Myh11-Cre:Acta2^{R179Hfl/+} mutant mice compared to Acta2^{R179Hfl/+} controls. Furthermore, Sapropterin-treated Myh11-Cre:Acta2^{R179Hfl/+} mutant mice exhibit a significantly restored average basal blood pressure compared to mutant Myh11-Cre:Acta2^{R179Hfl/+} mice.

FIGS. 11A-11B collectively show Colon and Small Intestine Measurements in Control vs. Mutant vs. Sapropterin-Treated Mutant Mice (FIG. 11A) The representative image illustrates the intestinal morphology, with Myh11-Cre:Acta2^{R179Hfl/+} mutant mice displaying fecal obstruction/impaction resulting from reduced intestinal motility. (FIG. 11B) Sapropterin-treated Myh11-Cre:Acta2^{R179Hfl/+} mutant mice show restored colon and small intestine length, indicating the efficacy of the treatment in alleviating the intestinal obstruction.

FIGS. 12A-12B Gut physiology experiments: Gut responses to electric field stimulation in control, mutant, and treated mutant mice. FIG. 12A: Representative traces illustrating the neuromuscular activity following electric field stimulation, as measured in control mice, mutant mice, and mutant mice treated with Sapropterin. FIG. 12B: The mutant mice with Myh11-Cre:Acta2^{R179Hfl/+} genotype treated with Sapropterin demonstrate a significantly restored contraction response to electric field stimulation when compared to the mutant mice with Myh11-Cre:Acta2^{R179Hfl/+} genotype.

FIGS. 13A-13C Hematoxylin and Eosin staining in mice brain section show differences in staining intensity in cortical regions in brain sections, between controls (FIG. 13A), mutants (FIG. 13B) and sapropterin treated mice (FIG. 13C). Mutant cortical section shows decrease number of cells and increased layering disarray compared to controls. Treatment with sapropterin show restoration of cell numbers and reduced cellular abnormality showing effectiveness of sapropterin treatment.

FIGS. 14A-14C Luxol Fast staining show differences in myelination stain intensity between controls (FIG. 14A), mutants (FIG. 14B) and sapropterin treated mice (FIG. 14C) in cortex, corpus callosum, striatum and internal capsule. Sapropterin treated mice section show improved myelination pattern compared to mutant mice in all brain regions.

### DETAILED DESCRIPTION

### Definitions and Abbreviations

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

For purposes of interpreting this specification, all of the functionalities described in connection with one embodiment of the methods, compositions, or kits described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

For purposes of interpreting this specification, the following definitions will apply and whenever appropriate, terms used in the singular will also include the plural and vice versa. As used in this specification and the appended claims, the singular terms “a,” “an” and “the” include plural referents unless the context clearly dictates otherwise.

Multisystemic smooth muscle dysfunction syndrome (abbreviated herein as MSMDS) (OMIM #613834) is an ultrarare genetic smooth muscle myopathy with major dysfunction in the vascular, respiratory, enteric, and genitourinary systems. Cases described to date have been monogenic, associated with the missense variation at arginine 179 of the ACTA2 gene, most commonly creating a missense variant (R179H). There are no pharmacological approaches, let alone treatment options, available for this disease.

Sapropterin, marketed as Kuvan®, is known chemically as 6R-(L-erythro)-5,6,7,8-tetrahydrobiopterin, of 6R-(L-erythro)-5,6,7,8-tetrahydrobiopterin (BH4) and has a chemical structure as shown below:

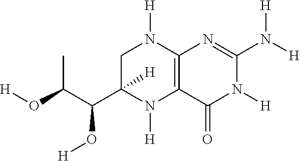

### Sapropterin

The term Sapropterin, as used herein, is also known by its IUPAC name as (6R)-2-amino-6-[(1R,2S)-1,2-dihydroxypropyl]-5,6,7,8-tetrahydro-3H-pteridin-4-one. Sapropterin may refer to a pharmaceutically acceptable salt. The term Sapropterin, as used herein, may refer to a pharmaceutically acceptable polymorph of a salt. Relevant salts include those formed with pharmacologically non-toxic acids, including mineral acids such as hydrochloric acid, phosphoric acid, sulfuric acid, boric acid; and organic acids such as acetic acid, formic acid, maleic acid, fumaric acid, mesylic acid. For example, the Sapropterin as used herein, may be Sapropterin hydrochloride, dihydrochloride or a polymorph thereof. For example, the Sapropterin may specifically refer to Sapropterin dihydrochloride or a pharmaceutically acceptable salt or/and polymorph thereof.

As used herein, a “subject” or “patient” is a vertebrate. In certain embodiments, the vertebrate is a mammal. Mammals include, but are not limited to, primates and rodents. In certain embodiments, a mammal is a human. Unless otherwise specified, a “subject”, as used herein, is meant to include a human fetus (e.g., in utero), infant, child, adolescent, adult. Unless otherwise specified, a subject or patient may refer to mammal less than 2 years of age, between 2 years and 5 years of age, between 5 and 10 years of age, between 10 and 18 years of age, or between about 18 and 30 years of age.

As used herein, “treating”, “treat” or “treatment” grammatical variations thereof, refers to clinical intervention in an attempt to alter the natural course of the individual having the genetic variation. Treating can be performed before development, as in “prophylactic treatment” for example in utero or during the course of clinical pathology. Desirable effects of treatment include preventing the occurrence of a disease or a condition or symptom thereof, alleviating a condition or symptom of the disease, diminishing any direct or indirect pathological consequences of the disease, decreasing the rate of disease progression, ameliorating or palliating the disease state, and/or achieving remission or improved prognosis.

An “effective amount” or “a therapeutically effective amount” unless otherwise indicated, refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the molecule to elicit a desired response in the individual. A therapeutically effective amount is also one in which any toxic or detrimental effects of the therapeutic agent are outweighed by the therapeutically beneficial effects.

The term “sample” or “biological sample”, as used herein, refers to a composition that is obtained or derived from a subject of interest that contains a genetic entity that is to be characterized and/or identified, for example using mutations detection methods. For example, the phrases “biological sample” refers to any sample obtained from a subject of interest that would be expected or is known to contain the genetic mutation that is to be characterized. Examples of samples include but are not limited to blood, solid tissue as from a fresh, frozen and/or preserved organ or tissue sample, biopsy, or aspirate; bodily fluids such as amniotic fluid, peritoneal fluid, or interstitial fluid; or cells from any time in gestation or development of the subject. The tissue sample may also be primary or cultured cells or cell lines.

The practice of the present invention will employ, unless otherwise indicated, conventional techniques of molecular biology (including recombinant techniques), microbiology, cell biology, biochemistry, physiology, histology, and immunohistochemistry, which are within the skill of the art. Such techniques are explained fully in the literature. In addition, primers, oligonucleotides and polynucleotides employed in the present invention can be generated using standard techniques known in the art. Unless defined otherwise, technical and scientific terms used herein have the same meaning as commonly understood by one of ordinary skill in the art to which this invention belongs.

### Method of Treatment, Kits and Formulations

Recent advances in electron cryomicroscopy have led to high resolution (3.7-4.7 Å) of structures of the actin filament. The inventors have discovered, using molecular dynamic simulation of two-four monomers, the presence of increased inter-subunit distances between two acta subunits (across the strand, between unit G and H) in ACTA2 deleterious substitution described herein. Each of these mutations have more than a 2-angstrom difference in distance compared with those measured in the normal actin fiber.

Next, the inventors screened tens of thousands of molecules, until the pharmacological drug Sapropterin, sold by the name of Kuvan®, was identified as capable of functionally compensating for the defective actin filament resulting from the substitution mutations in the ACTA2 gene. It was initially proposed and subsequently validated in animal models that Sapropterin can return the native structure to the mutant actinA2 filament.

In an animal model, Sapropterin was made orally available for pregnant mom throughout pregnancy as well as postnatally. Analysis of treated Myh11-Cre:Acta2^{R179Hfl/+} mice revealed restoration of the phenotype of the R179H mutation. Importantly, the Sapropterin treated mice, exhibited a dramatic phenotypic recovery, including improved weight gain, rotarod and open field exploratory and locomotive behavior performance, improved GI physiology, and extended lifespan.

Thus, the present invention, based on discovering specific amino acid variations in ACTA2 protein which the inventors have now associated with treatment by sapropterin, provides products, combinations, kits and methods involving Sapropterin (i.e., tetrahydrobiopterin) for use in a patient diagnosed with MSMDS or a subject at risk for developing MSMDS.

In a first aspect, the present invention provides a method of treating an MSMDS patient or a subject having or suspected of having a substitution in an actin alpha (ACTA2) locus, comprising administering a therapeutically effective amount of Sapropterin to the patient or subject, contingent upon the presence of a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S). In some embodiments, there is provided a method of treating a subject in a risk category for developing MSMDS comprising: obtaining a biological sample from the subject, detecting in the biological sample a presence of the abovementioned substitution in an actin alpha (ACTA2) locus; and indicating of a propensity for an increased likelihood of pharmacological effectiveness or benefit of treatment of sapropterin.

The actin alpha (ACTA2) protein refers to SEQ 1 as listed below, while position R179 is underlined and bolded and represented by “R”:
sp|P62736|ACTA_HUMAN Actin, aortic smooth muscle OS=Homo sapiens OX=9606 GN=ACTA2 PE=1; SV=1

SEQ ID NO. 1
MCEEEDSTALVCDNGSGLCKAGFAGDDAPRAVFPSIVGRPRHQGVMVGM
GQKDSYVGDEAQSKRGILTLKYPIEHGIITNWDDMEKIWHHSFYNELRV
APEEHPTLLTEAPLNPKANREKMTQIMFETFNVPAMYVAIQAVLSLYAS
GRTTGIVLDSGDGVTHNVPIYEGYALPHAIMRLDLAGRDLTDYLMKILT
ERGYSFVTTAEREIVRDIKEKLCYVALDFENEMATAASSSSLEKSYELP
DGQVITIGNERFRCPETLFQPSFIGMESAGIHETTYNSIMKCDIDIRKD
LYANNVLSGGTTMYPGIADRMQKEITALAPSTMKIKIIAPPERKYSVWI
GGSILASLSTFQQMWISKQEYDEAGPSIVHRKCF

Consistent with some disclosed embodiments, the subject is diagnosed with or in a risk category for developing a Multisystemic smooth muscle dysfunction syndrome (MSMDS).

Consistent with some disclosed embodiments, treatment may refer to any benefit from treatment and may include in this context, prevention, delaying development and/or reducing progression of an R179H mutation-driven actin disease by administering a therapeutically effective amount of Sapropterin. Treatment may refer to predicting sensitivity or indicative of a propensity for an increased likelihood of pharmacological effectiveness or benefit of treatment. For example, the methods, and compositions of the invention may be useful in attempt to delay the development of a disease or disorder.

As used herein, a therapeutically effective amount may refer to any therapeutically beneficial response elicited by administration. A beneficial response may include an amount sufficient to prevent, delay development or reduce the rate of progression. A “response” of a subject, can be assessed using any endpoint indicating a benefit to the patient, including, without limitation: inhibition, to some extent, of disease progression, slowing down and complete arrest; relief, to some extent, of one or more symptoms associated with the disorder; increase in a length of progression of disease following treatment; decreased mortality at a given point of time following treatment; and/or an increase in functional F-actin, an increase in a ratio between F-actin to G-actin, or a decrease in functional G-actin. For example, a therapeutically effective amount may refer to normalization on the cellular level, of G/F actin ratio as detected by Western blot and/or cell immunofluorescence.

Consistent with some disclosed embodiments, there is provided a method for predicting sensitivity to treatment or indicative of a propensity for an increased likelihood of pharmacological effectiveness or benefit of treatment of Sapropterin in a subject diagnosed with or in a risk category for developing MSMDS based on a genetic substitution in the actin alpha (ACTA2) locus wherein the genetic substitution results in an amino acid change, at position 179, of arginine for histidine (R179H), leucine (R179L), cysteine (R179C), glycine (R179G) or serine (R179S).

Consistent with some disclosed embodiments, a therapeutically effective amount of Sapropterin may refer to an amount sufficient to restore function of cells harboring the ACTA2 R179H pathogenic variant. This was demonstrated in a mouse model of multisystemic smooth muscle dysfunction syndrome where the drug was introduced into the drinking water at the time of mating and replaced twice a week. Continued access to sapropterin ensures peri and postnatal exposure to the drug for newborn mice until the weaning age. Post weaning, the drug was placed separately in the weaned cages, and the weaned animals were given sapropterin until the end of life or experiments. A therapeutically effective amount of Sapropterin may refer to an amount sufficient to improve smooth muscle function in the gastrointestinal tract, cardiovascular system, and/or blood vessels.

A therapeutically effective amount of Sapropterin may refer to a dosage of 10 to 30 mg/kg.

Based on the discovery that sapropterin-treated R179H mutant fibroblasts show significant improvements of polymerized fibrillary actin (F-actin) levels compared to untreated mutant and wild-type fibroblasts, a therapeutically effective amount may be an amount sufficient to restore alpha smooth muscle actin (SMA) polymerization in human patient-derived fibroblasts with the R179H mutation. For example, it may be an amount sufficient to cause in the subject, subsequent to treatment, any one or more of the following: an increase in functional F-actin; an increase in a ratio between F-actin (fibrous actin polymerized in the form of a double helix) to G-actin (globular monomeric form); and degradation or decrease G-actin formation.

Consistent with some disclosed embodiments, the therapeutically effective amount of sapropterin may refer to administering a prophylactically effective amount. A “prophylactically effective amount” may refer to an amount effective, at dosages and for periods of time necessary, to achieve the desired prophylactic result. Typically, but not necessarily, since a prophylactic dose is used in subjects prior to or at an earlier stage of disease, the prophylactically affective amount will often be less than the amount necessary to restore dysfunctional actin alpha (ACTA2).

Consistent with some disclosed embodiments, the sapropterin may be administered by intravenous administration, as a bolus or by continuous infusion over a period of time, by intramuscular, intraperitoneal, intracerebrospinal, subcutaneous, intra-articular, intrasynovial, intrathecal, oral, topical, or inhalation routes. Consistent with some disclosed embodiments, administering refers to orally administering. In some embodiments, the administering a therapeutically effective amount of Sapropterin to a subject is by systemic administration. In some embodiments, the administering a therapeutically effective amount of Sapropterin to a subject is by local administration. In some embodiments, the administering a therapeutically effective amount of Sapropterin to a subject is by providing ad-libitum access drinking water with Sapropterin.

Consistent with some disclosed embodiments, the subject is a fetus, a child, or an adult. For example, the child may be less than one-year old.

Consistent with some disclosed embodiments, there is provided a congenital treatment of MSMDS by Sapropterin. In this case, the subject may be a fetus. Consistent with some disclosed embodiments, a subject being a fetus may be suspected of having the genetic variation based on a suspicious medical result or alternatively, as part of a battery of genetic testing typical prior to brith.

In this embodiment, administering a therapeutically effective amount of sapropterin may involve administering to a pregnant adult thereby exposing or administering indirectly to a developing fetus. For example, early indicators may prompt genetic testing, which is common practice during pregnancy, and provide opportunity to influence development of the phenotype.

Consistent with some disclosed embodiments, formulations of the present invention may be suitable for oral administration and provided in the form of tablets, sublingual tablets, capsules, powders, granules or fine granules, or suspensions in a non-aqueous liquid such as emulsions, potions or syrups, that contain the prescribed amount of one or more active ingredients.

Consistent with some disclosed embodiments, administering a therapeutically effective amount of Sapropterin to a subject, is contingent upon the presence of one or more of a genetic substitution in the actin alpha (ACTA2) locus. The genetic variation in the Actin Alpha 2 gene may be the presence of a minor allele of at least one single nucleotide polymorphism (SNP) of rs387906592. A select list of relevant substitutions in the actin alpha (ACTA2) locus may be a genetic substitution which results in an amino acid change, at position 179, of arginine for histidine (R179H), leucine (R179L), cysteine (R179C), glycine (R179G) or serine (R179S). Said another way, the genetic substitution in the Actin Alpha 2 locus maybe be a genetic substitution which results in an amino acid change being a histidine (H), leucine (L), glycine (G), a cysteine (C), serine(S) allele of a single nucleotide polymorphism of rs886039303.

Consistent with some disclosed embodiments, contingent upon refers to detecting a positive confirmation of the presence of a genetic variation. The presence of the genetic variations may be provided by: obtaining a biological sample from the subject and detecting in the biological sample a presence of a genetic substitution which results in an amino acid change, at position 179, of arginine for histidine (R179H), leucine (R179L), cysteine (R179C), glycine (R179G) or serine (R179S). Detecting may refer to identifying in the genetic or protein material. Detecting may refer to analyzing the nucleic acid sequence for allelic polymorphism which results in an amino acid change at position 179, of arginine for histidine (R179H), leucine (R179L), cysteine (R179C), glycine (R179G) or serine (R179S).

The term “detection” includes any means of detecting, including direct and indirect detection. Variations may be detected by certain methods known to those skilled in the art. Such methods include, but are not limited to, primer extension assay such as allele-specific primer extension assays; nucleotide incorporation assay such as allele-specific nucleotide incorporation assays such as single base extension assays; oligonucleotide hybridization assay such as allele-specific oligonucleotide hybridization assays (e.g., oligonucleotide ligation assays); a 5′ nuclease assay; an assay employing molecular beacons; DNA sequencing, genetic bit analysis (GBA) and an oligonucleotide ligation assay.

Detection of variations in target nucleic acid sequence may be accomplished by amplification or molecular cloning and sequencing of the target nucleic acids using techniques well known in the art. The term “amplification” refers to the process of producing one or more copies of a reference nucleic acid sequence or its complement. Amplification may be linear or exponential. The term “target sequence” or “target nucleic acid sequence” refers generally to a polynucleotide sequence of interest in which a nucleotide variation is suspected or known to reside, including copies of such target nucleic acid generated by amplification. Amplification may be desirable in certain instances, e.g., in order to obtain a desired amount of material for detecting variations. The amplicons may then be subjected to a detection method, such as those described below, to determine whether a variation is present in the amplicon.

The nucleic acid sequence of the amplified sequences can then be determined. A nucleic acid is said to be “derived from” a particular source if it is obtained directly from that source or if it is a copy of a nucleic acid found in that source such as a copy that result from amplification. Nucleic acid, as used herein, may be genomic DNA, RNA transcribed from genomic DNA, or cDNA generated from RNA.

In another aspect, there is provided a Saproptrin or a pharmaceutically acceptable salt or polymorph thereof, for use in the manufacture of a formulation for the treatment of a subset of subjects diagnosed with or in a risk category for developing MSMDS based on a genetic variation previously mentioned. Formulations of the present invention are prepared by formulating any of the molecules listed in the present disclosure with a pharmaceutically acceptable carrier by conventional procedures into a dosage form suitable for oral, rectal, or parenteral administration. Depending on the dosage form chosen, the carrier used for these pharmaceutical formulations generally includes excipients, binders, disintegrants, antioxidants, buffers, flavors, surfactants, thickeners, lubricants, or any other conventional excipients.

Single or multiple dosages may be employed. For example, a therapeutically effective dosage or amount of Sapropterin may be used alone and may range from about 1 mg/kg to about 20 mg/kg of body weight or more per day. Interspecies scaling of dosages can be performed in a manner known in the art. When in vivo administration of Sapropterin is employed, normal dosage amounts may vary from about 3 ng/kg to up to 7 mg/kg of mammal body weight or more per day, preferably about 5 mg/kg/day. A desired dosage of Sapropterin may be administered once a day or may be administered in divided doses of two to four times a day at appropriate intervals.

The Sapropterin may be administered alone without being mixed with other ingredients, or as a formulation containing other active ingredients or excipients. For example, in accordance with aspects and embodiments of the disclosure, the Sapropterin may be formulated in a variety of different ways to promote treatment. The formulation may also include excipients which play a role in formulation, stabilization or contribute to manufacturing of the active agent.

Consistent with some disclosed embodiments, administering a therapeutically effective amount of sapropterin may be separately, sequentially, or simultaneously with an anti-platelet (aspirin, placid), anticoagulant (LMWH, heparin or oral anti-Xa agents), anti-epileptic, or inhaled active.

The present disclosure, in accordance with aspects and embodiments herein, may include a detection kit. The kit could include all components necessary for performing any one or more of the following assays: primer extension assay such as allele-specific primer extension assays; nucleotide incorporation assay such as allele-specific nucleotide incorporation assays such as single base extension assays; oligonucleotide hybridization assay such as allele-specific oligonucleotide hybridization assays (e.g., oligonucleotide ligation assays); a 5′ nuclease assay; an assay employing molecular beacons; DNA sequencing, genetic bit analysis (GBA) and an oligonucleotide ligation assay. The kit may also include specific primers, oligonucleotide sequences, molecular beacon, or GBA which are specific to the genetic variations listed in the present invention.

Kits will typically include the necessary components described above as well as materials desirable from a commercial and user standpoint, including buffers (e.g., block buffer, wash buffer, substrate buffer, etc.), diluents, filters, needles, syringes, and package inserts with instructions for use. A label may be present on the container to indicate that the composition is used for a specific diagnosis or therapy.

In another aspect, there is provided a kit comprising components for detecting in a biological sample obtained from a subject, a presence of a substitution in an actin alpha 2 (ACTA2) gene wherein the substitution in an actin alpha (ACTA2) locus is, at position 179, a substitution of arginine for histidine (R179H), leucine (R179L), cysteine (R179C), glycine (R179G) or serine (R179S).

In another aspect, there is provided a method for manufacturing a formulation comprising Sapropterin for treatment of a subset of subjects diagnosed with or in a risk category for developing a MSMDS comprising: packaging the Sapropterin formulation with instructions to administer the formulation to a subject selected based on detecting in a biological sample of the subject of one or more nucleotide substitutions selected from: a substitution that results in an amino acid change consisting of a substitution in an actin alpha (ACTA2) locus is selected from one or more of the following: at position 179, a substitution of arginine for histidine (R179H), leucine (R179L), cysteine (R179C), glycine (R179G) or serine (R179S). In one example, the nucleotide substitution which results in the a substitution of arginine for histidine at position 179 (R179H) or a histidine (H) allele of a single nucleotide polymorphism SNP of rs387906592.

While the invention has been disclosed in this patent application by reference to the details of various embodiments of the invention, it is to be clear that the disclosure is intended in an attempt to illustrate rather than to be limiting. It is contemplated that modifications will readily occur to those skilled in the art, within the spirit of the invention and the scope of the appended claims.

### EXAMPLES

### Example 1: Mouse and Cellular Models of ACTA2 R179H MSMDS

A mouse models and several cellular models of MSMDS, including a conditional knock-in Acta2R179H mouse that when crossed with a Myh11-Cre mouse develop the characteristic SMC systemic dysregulation and early onset of aortic dilatation, cerebral steno-occlusive vasculopathy and BBB dysfunction. Also described is an ACTA2 R179H dermal fibroblast cell-line that permits evaluation of corrective pharmacological strategies. These cellular models were used to demonstrate effective correction of the ACTA2 179 codon from the disease causing ‘CAT’ codon to the wild type ‘CGT,’ allele-specific knockout of the R179H allele using drug therapy.

Provided herein are conditional knock in ACTA2 Arg179His mouse models, the somatic and germ line cells of which include a mutant ACTA2 exon 6. The wild type allele is with a poly A terminator is surrounded by loxP sites. When the mice are crossed with a mouse expressing a Cre recombinase the wild type exon is removed and the mutant exon is spliced into the ACTA2 mRNA allele, resulting in expression of a mutant alpha smooth muscle actin within cell types expressing a Cre recombinase. For example, to activate the R179H allele in smooth muscle containing organs a cre recombinase driven by a smooth muscle cell myosin heavy chain promoter (Myh11-cre) can be used.

Altered Actin and Cytoskeleton in Patients with ACTA2 R179 Mutations

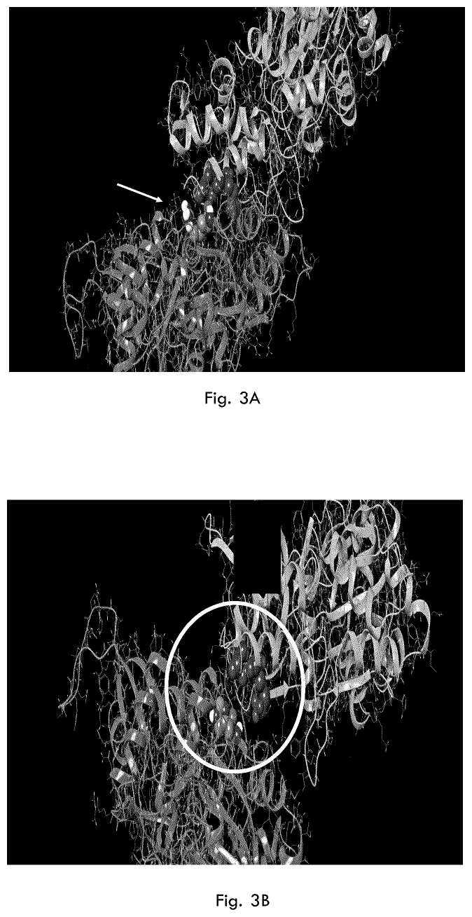

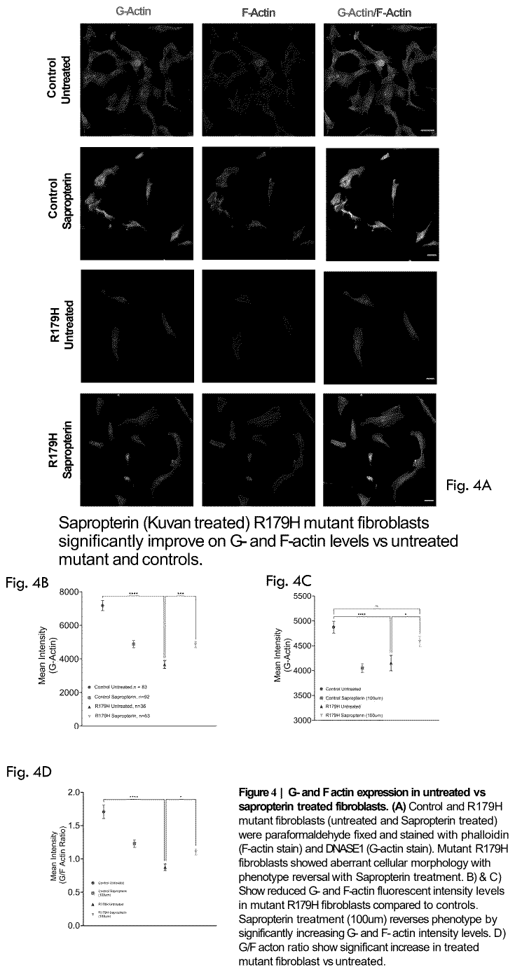

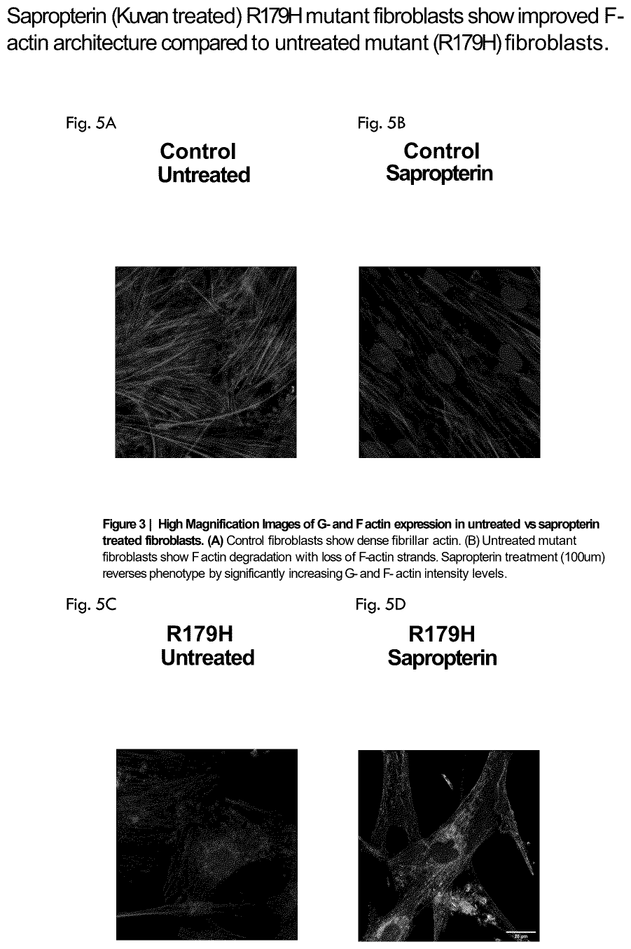

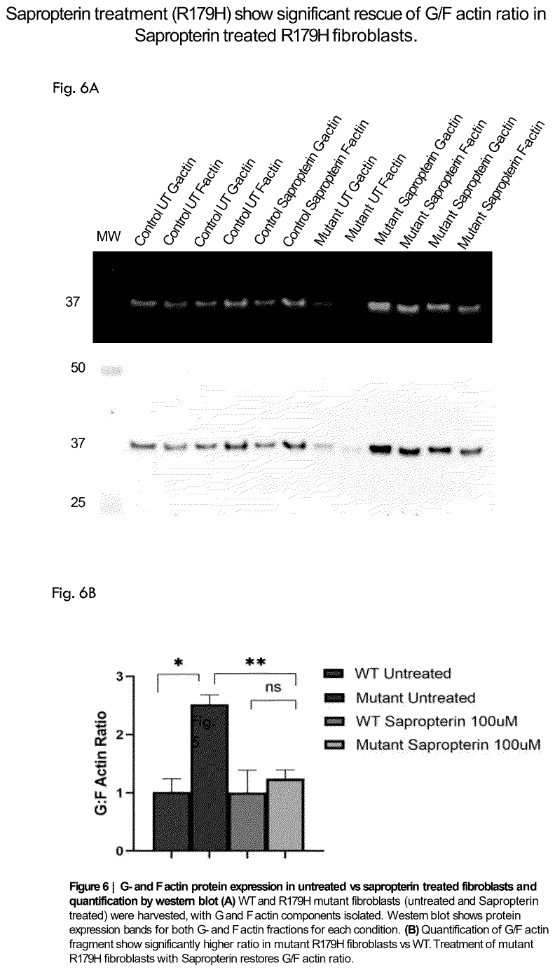

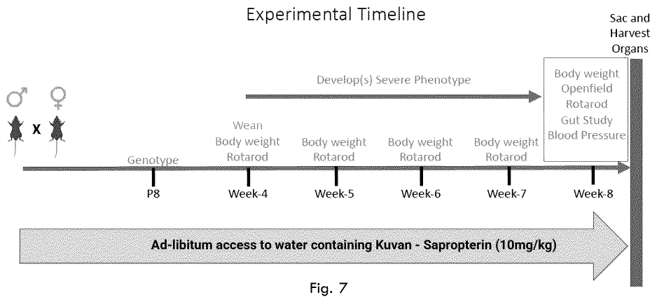

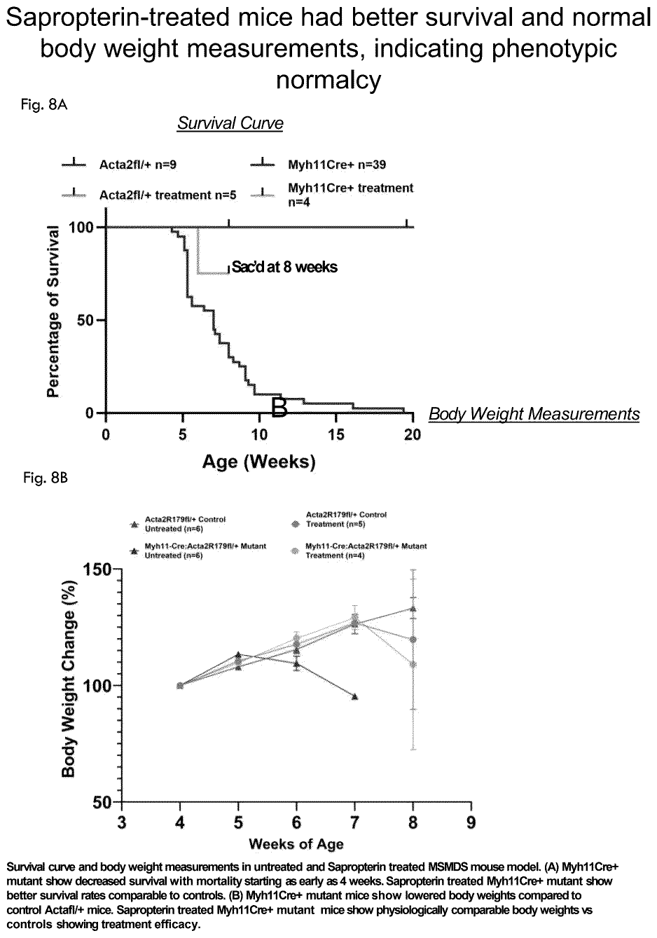

In this experiment, human skin fibroblasts were grown from a patient with an ACTA2 R179H mutation and a control (unaffected) subjects. Cell contents were fractionated through centrifugation to separate polymerized filamentous beta actin (F-actin) from globular beta actin (G-actin). Beta actin was identified via western blot with antibody directed against it (Cytoskeleton Inc., Clone 7A8.2.1). The results, shown in FIG. 2, demonstrate that cells expressing the mutant ACTA2 proteins have a decreased amount of polymerized (F-actin) and altered ratios of G/F actin in the affected subjects.

In addition, the same cells were stained for the presence of polymerized beta-actin (F-actin) with phalloidin-FITC and an α-smooth muscle actin antibody, and the cell nuclei were stained with DAPI.

The invention is further described in the following examples, which do not limit the scope of the invention described in the claims.

### Materials and Methods

The following materials and methods were used in examples described herein.

To characterize the neurovascular and systemic features of MSMDS we created a knock in mouse expressing a mutant ACTA2 with a missense mutation at position 179 replacing arginine with histidine (Acta2^{R179fl/+}) under the control of tissue-specific cre excision. Herein, we characterize the multisystemic features of the animal model and an extensive and neurovascular evaluation to gain insight into the human syndrome.

Creation of ACTA2^{R179fl+} knock in mouse—To characterize the neurovascular features of MSMDS a Cre-inducible ACTA2^{R179H} knock in mouse was created. The mouse contains a mutant exon 6 downstream of a polyA terminator and surrounded by loxP sites. Cre mediated excision advances the mutant exon to be spliced into the ACTA2 mRNA allele, resulting in expression of a mutant alpha smooth muscle actin within cell types expressing a Cre recombinase. To activate the R179H allele in multiple smooth muscle containing organs we used cre recombinase driven by a smooth muscle cell myosin heavy chain cre (Myh11-cre) and to activate the allele within the anterior cerebral circulation without other systemic effects, we used the neural crest specific cre allele (Wnt1-cre)²⁰. Systemically, Myh11-Cre:Acta2^{R179fi/+} mice were smaller, gained weight slower, and had early mortality when compared to either Wnt1-Cre:Acta2^{R179fl/+} or Acta2^{R179fl/+} littermates. Other phenotypes reminiscent of MSMDS were notable including abdominal distention with thinning of the abdominal wall suggestive of Prune-Belly syndrome and a primary pulmonary emphysema, both of which have been seen in human patients.

Gross Organ images: For gastrointestinal morphology study, GI of mice were isolated and perfused with PBS and photographed for gross visualization. The entire GI tract were isolated and perfused with PBS. A small section of the desired GI tract were used with a force transducer equipment to record gut motility and integrity in response to various physiological stimuli like chemical and electrical triggers. Contraction force of the gut was assessed and efficacy of Sapropterin treatment was calculated in mutant mice. Histology: Organs were then removed from the animals or dissected in situ for photography prior to paraffinization and sectioning (7 μM). Slides were produced for tissue staining or stained with standard stains including Hematoxylin & Eosin, Elastin (Verhoeff-Van Gieson and Luxol Fast Blue for quantitative analysis

### G- and F-Actin Measurements in Human Control and Mutant Fibroblasts:

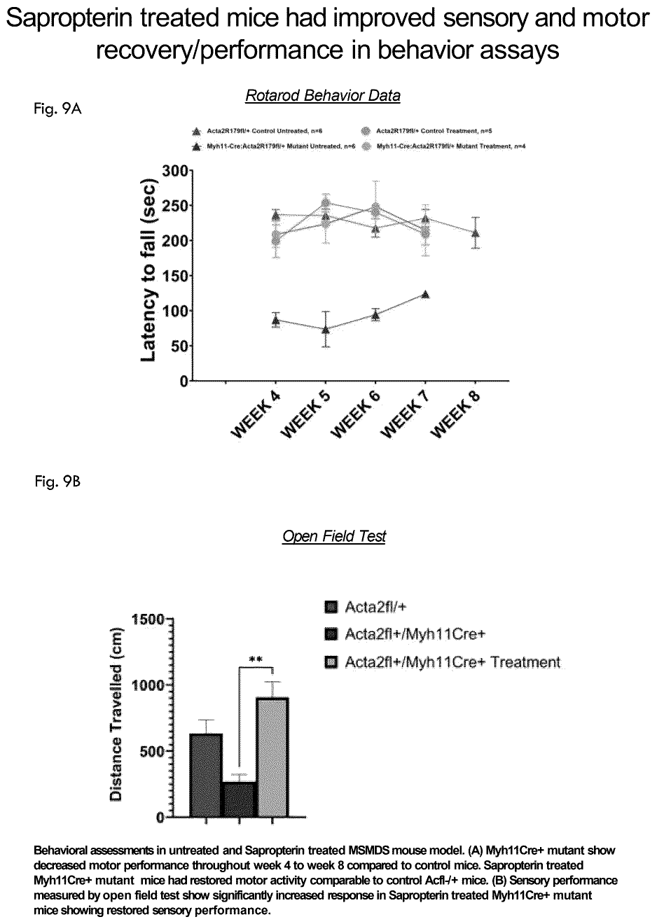

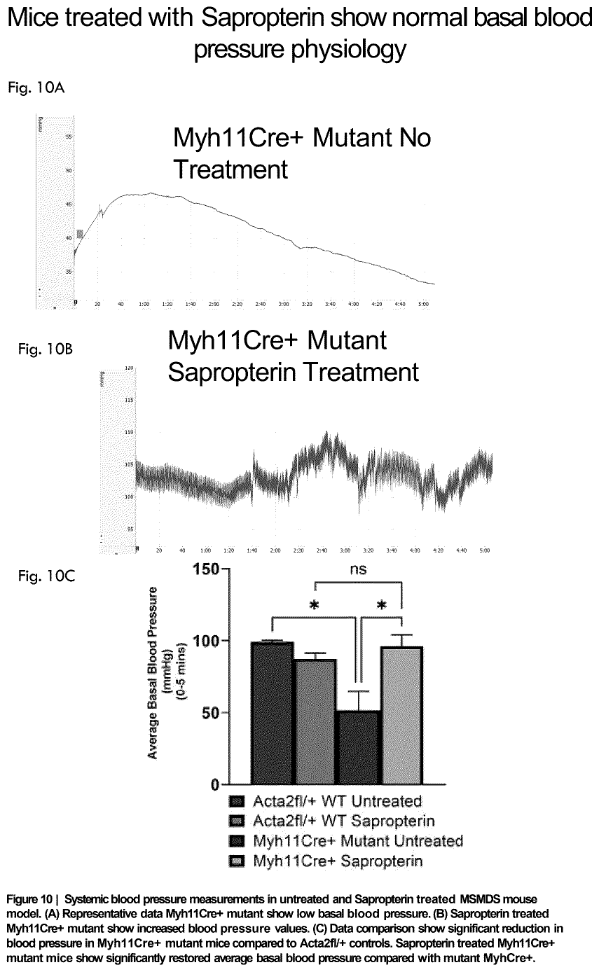

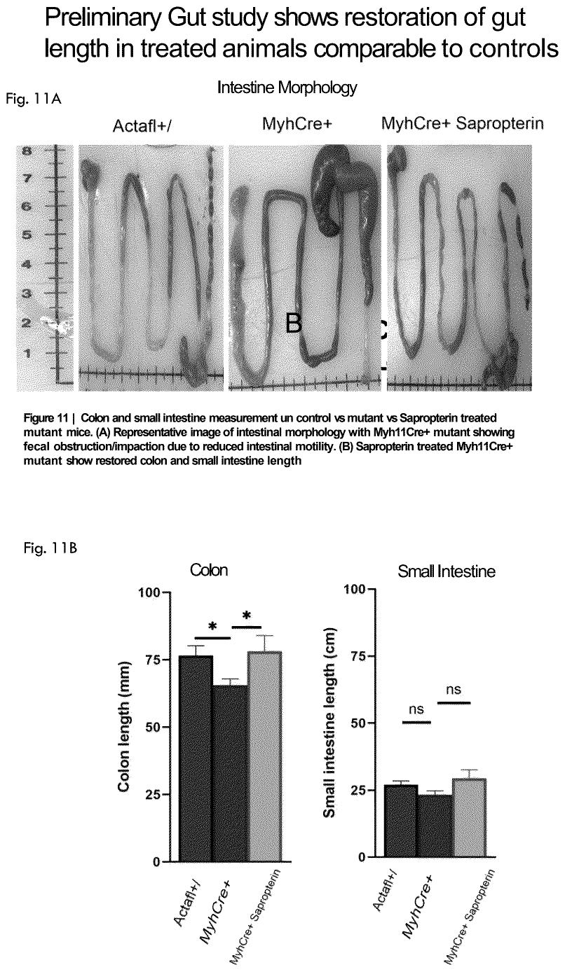

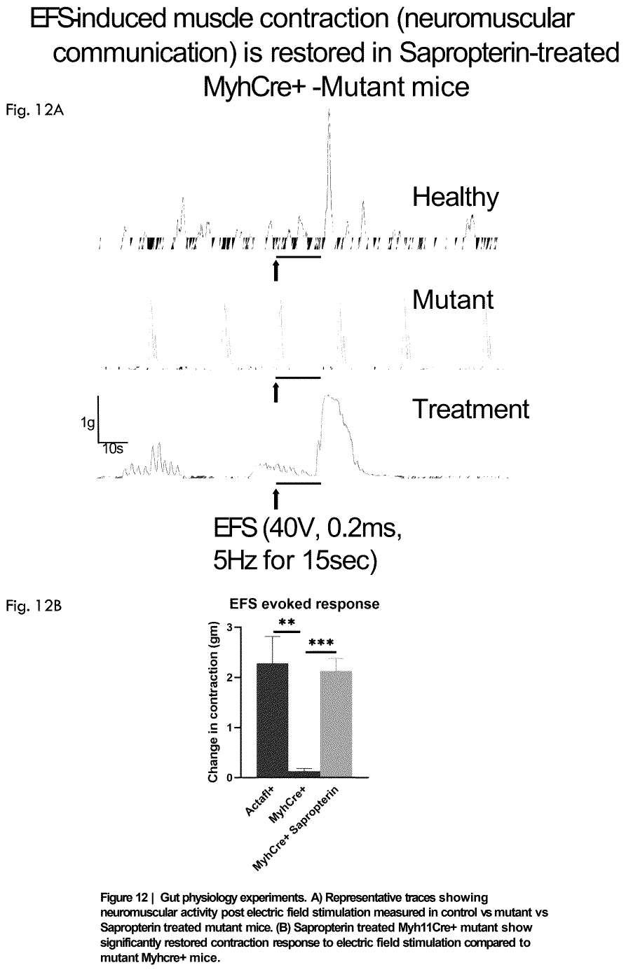

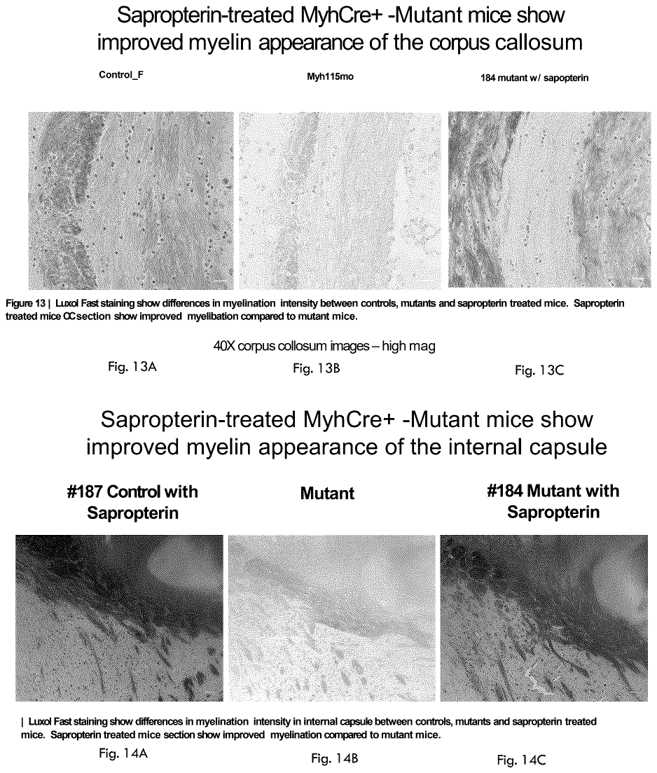

Human skin fibroblasts were grown from a patient with an ACTA2 R179H mutation, a patient with ACTA2 R179C mutation, and a control (unaffected) subjects. Cell contents were fractionated through centrifugation to separate polymerized filamentous beta actin (F-actin) from globular beta actin (G-actin). Beta actin was identified via western blot with antibody directed against it (Cytoskeleton Inc., Clone 7A8.2.1). The results, shown in FIG. 9, demonstrate that cells expressing the mutant ACTA2 proteins have a decreased amount of polymerized (F-actin) and altered ratios of G/F actin in the affected subjects.

Blood pressure measurement: Blood pressures (BPs) were measured via femoral artery catheter (MacLab; ADInstruments, Colorado Springs, MO) under general anesthesia. During the procedure, mice were anesthetized under isoflurane 3% induction and 1-1.5% maintenance in 70% N₂O and 30% O₂. Mice temperature was maintained at 37° C. using rectal temperature and a heating pad. The left femoral artery was catheterized with heparinized PE10 tubing. After cannulation, the animals were waiting for stabilize BPs for minutes. After stabilization, arterial pressure (mmHg) was recorded (PowerLab, ADInstruments, Colorado Springs, CO, USA), and an arterial blood samples were drawn to measure pH, partial pressure oxygen (pO₂), partial pressure of carbon dioxide (pCO₂) at the same time that diffuse correlation spectroscopy (DCS) measurements were done. Systolic BPs (SBPs), means BPs (MBPs) and dilated BPs (DBPs) were assessed using stabilized value (Buckley et al., J Cereb Blood Flow Metab. 2015 December; 35 (12): 1995-2000; Behavior: AnyMaze software (ver. 8.42, Stoelting, Wood Dale, IL, USA) was used for tracking and analysis. For the Open field testing (OFT), mice were placed in a 28×18 cm open field. Distance traveled and speed were recorded for 30 min. The apparatus was cleaned with 70% ethanol between trials. For the rotarod, five trials were performed each day for three days. Mice were placed on a rod at a starting rotation of 4 r/min which was constantly accelerated to 40 r/min over the course of 120 s. The latency of falling off the rod and the rod RPMs were recorded.

### REFERENCES

1. Jinek, M. et al. A Programmable Dual-RNA-Guided DNA Endonuclease in Adaptive Bacterial Immunity. Science 337, 816-821 (2012).
2. Doudna, J. A. The promise and challenge of therapeutic genome editing. Nature 578, 229-236 (2020).
3. Sternberg, S. H., Redding, S., Jinek, M., Greene, E. C. & Doudna, J. A. DNA interrogation by the CRISPR RNA-guided endonuclease Cas9. Nature 507, 62-67 (2014).
4. Kleinstiver, B. P. et al. Engineered CRISPR-Cas9 nucleases with altered PAM specificities. Nature 523, 481-485 (2015).
5. Kleinstiver, B. P. et al. Broadening the targeting range of Staphylococcus aureus CRISPR-Cas9 by modifying PAM recognition. Nat. Biotechnol. 33, 1293-1298 (2015).
6. Kleinstiver, B. P. et al. High-fidelity CRISPR-Cas9 nucleases with no detectable genome-wide off-target effects. Nature 529, 490-495 (2016).
7. Kleinstiver, B. P. et al. Engineered CRISPR-Cas12a variants with increased activities and improved targeting ranges for gene, epigenetic and base editing. Nat. Biotechnol. 37, 276-282 (2019).
8. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
9. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
10. Gaudelli, N. M. et al. Programmable base editing of A.T to G.C in genomic DNA without DNA cleavage. Nature 551, 464-471 (2017).
11. Rees, H. A. & Liu, D. R. Base editing: precision chemistry on the genome and transcriptome of living cells. Nat. Rev. Genet. 19, 770-788 (2018).
12. Porto, E. M., Komor, A. C., Slaymaker, I. M. & Yeo, G. W. Base editing: advances and therapeutic opportunities. Nat. Rev. Drug Discov. 19, 839-859 (2020).
13. Anzalone, A. V. et al. Search-and-replace genome editing without double-strand breaks or donor DNA. Nature 576, 149-157 (2019).
14. Anzalone, A. V., Koblan, L. W. & Liu, D. R. Genome editing with CRISPR-Cas nucleases, base editors, transposases and prime editors. Nat. Biotechnol. 38, 824-844 (2020).
15. Gaudelli, N. M. et al. Directed evolution of adenine base editors with increased activity and therapeutic application. Nat. Biotechnol. 38, 892-900 (2020).
16. Richter, M. F. et al. Phage-assisted evolution of an adenine base editor with improved Cas domain compatibility and activity. Nat. Biotechnol. 38, 883-891 (2020).
17. Kleinstiver, B. & Walton, R. T. Crispr-cas enzymes with enhanced on-target activity. US2021/0261932
18. Miller, S. M. et al. Continuous evolution of SpCas9 variants compatible with non-G PAMs. Nat. Biotechnol. 38, 471-481 (2020).
19. Levy, J. M. et al. Cytosine and adenine base editing of the brain, liver, retina, heart and skeletal muscle of mice via adeno-associated viruses. Nat. Biomed. Eng. 4, 97-110 (2020).
20. Wu, H., Hu, Z. & Liu, X.-Q. Protein trans-splicing by a split intein encoded in a split DnaE gene of Synechocystis sp. PCC6803. Proc. Natl. Acad. Sci. 95, 9226-9231 (1998).
21. Zettler, J., Schütz, V. & Mootz, H. D. The naturally split Npu DnaE intein exhibits an extraordinarily high rate in the protein trans-splicing reaction. FEBS Lett. 583, 909-914 (2009).
22. Stevens, A. J. et al. Design of a Split Intein with Exceptional Protein Splicing Activity. J. Am. Chem. Soc. 138, 2162-2165 (2016).
23. Koblan, L. W. et al. Improving cytidine and adenine base editors by expression optimization and ancestral reconstruction. Nat. Biotechnol. 36, 843-846 (2018).
24. Rohland, N. & Reich, D. Cost-effective, high-throughput DNA sequencing libraries for multiplexed target capture. Genome Res. 22, 939-946 (2012).
25. Clement, K. et al. CRISPResso2 provides accurate and rapid genome editing sequence analysis. Nat. Biotechnol. 37, 224-226 (2019).
26. Munot, P., et al. A novel distinctive cerebrovascular phenotype is associated with heterozygous Arg179 ACTA2 mutations. Brain 135, 2506-2514 (2012).
27. Regalado, E. S., et al. Clinical history and management recommendations of the smooth muscle dysfunction syndrome due to ACTA2 arginine 179 alterations. Genet Med 20, 1206-1215 (2018).
28. Etchevers, H. C., Vincent, C., Le Douarin, N. M. & Couly, G. F. The cephalic neural crest provides pericytes and smooth muscle cells to all blood vessels of the face and forebrain. Development 128, 1059-1068 (2001).
29. Jiang, X., et al. Normal fate and altered function of the cardiac neural crest cell lineage in retinoic acid receptor mutant embryos. Mech Dev 117, 115-122 (2002).
30. Li, Y., et al. Intrastriatal transplantation of bone marrow nonhematopoietic cells improves functional recovery after stroke in adult mice. J Cereb Blood Flow Metab 20, 1311-1319 (2000).
31. Chung, D. Y., et al. Determinants of Optogenetic Cortical Spreading Depolarizations. Cereb Cortex (2018).
32. Xie, H., et al. Differential effects of anesthetics on resting state functional connectivity in the mouse. J Cereb Blood Flow Metab 40, 875-884 (2020).
33. Ash, J. A., et al. Functional connectivity with the retrosplenial cortex predicts cognitive aging in rats. Proc Natl Acad Sci USA 113, 12286-12291 (2016).
34. Kitagawa, K., et al. Cerebral ischemia after bilateral carotid artery occlusion and intraluminal suture occlusion in mice: evaluation of the patency of the posterior communicating artery. J Cereb Blood Flow Metab 18, 570-579 (1998).
35. Hattori, Y., Kitamura, A., Nagatsuka, K. & Ihara, M. A novel mouse model of ischemic carotid artery disease. PloS one 9, e100257 (2014).
36. Papke, C. L., et al. Smooth muscle hyperplasia due to loss of smooth muscle alpha-actin is driven by activation of focal adhesion kinase, altered p53 localization and increased levels of platelet-derived growth factor receptor-beta. Hum Mol Genet 22, 3123-3137 (2013).
37. Lino Cardenas, C. L., et al. An HDAC9-MALATI-BRG1 complex mediates smooth muscle dysfunction in thoracic aortic aneurysm. Nat Commun 9, 1009 (2018).
38. Schiffner, R., et al. Redistribution of Cerebral Blood Flow during Severe Hypovolemia and Reperfusion in a Sheep Model: Critical Role of alpha1-Adrenergic Signaling. Int J Mol Sci 18 (2017).
39. Grunstein, M. Histone acetylation in chromatin structure and transcription. Nature 389, 349-352 (1997).
40. Haberland, M., Montgomery, R. L. & Olson, E. N. The many roles of histone deacetylases in development and physiology: implications for disease and therapy. Nat Rev Genet 10, 32-42 (2009).
41. Kato, N., et al. Trans-ancestry genome-wide association study identifies 12 genetic loci influencing blood pressure and implicates a role for DNA methylation. Nat Genet 47, 1282-1293 (2015).
42. Foroud, T., et al. Genome-wide association study of intracranial aneurysm identifies a new association on chromosome 7. Stroke; a journal of cerebral circulation 45, 3194-3199 (2014).
43. International Stroke Genetics, C., et al. Genome-wide association study identifies a variant in HDAC9 associated with large vessel ischemic stroke. Nat Genet 44, 328-333 (2012).
44. Myocardial Infarction Genetics, C., et al. Genome-wide association of early-onset myocardial infarction with single nucleotide polymorphisms and copy number variants. Nat Genet 41, 334-341 (2009).
45. Lino Cardenas, C. L., et al. An HDAC9-MALATI-BRG1 complex mediates smooth muscle dysfunction in thoracic aortic aneurysm. Nat Commun 9, 1009 (2018).
46. Gallo, E. M., et al. Angiotensin II-dependent TGF-beta signaling contributes to Loeys-Dietz syndrome vascular pathogenesis. J Clin Invest 124, 448-460 (2014).
47. Regalado, E. S., et al. Aortic Disease Presentation and Outcome Associated with ACTA2 Mutations. Circulation. Cardiovascular genetics 8, 457-464 (2015).
48. Lino Cardenas, C. L., et al. HDAC9 complex inhibition improves smooth muscle-dependent stenotic vascular disease. JCI Insight 4 (2019).
49. Haberland, M., et al. Regulation of HDAC9 gene expression by MEF2 establishes a negative-feedback loop in the transcriptional circuitry of muscle differentiation. Mol Cell Biol 27, 518-525 (2007).
50. Judge, D. P., et al. Evidence for a critical contribution of haploinsufficiency in the complex pathogenesis of Marfan syndrome. J Clin Invest 114, 172-181 (2004).
51. Silasi, G., Xiao, D., Vanni, M.P., Chen, A. C. & Murphy, T. H. Intact skull chronic windows for mesoscopic wide-field imaging in awake mice. J Neurosci Methods 267, 141-149 (2016).
52. Xie, H., et al. Differential effects of anesthetics on resting state functional connectivity in the mouse. J Cereb Blood Flow Metab 40, 875-884 (2020).
53. White, B. R., et al. Imaging of functional connectivity in the mouse brain. PloS one 6, e16322 (2011).
54. Kura, S., et al. Intrinsic optical signal imaging of the blood volume changes is sufficient for mapping the resting state functional connectivity in the rodent cortex. J Neural Eng 15, 035003 (2018).
55. Edelstein, A. D., et al. Advanced methods of microscope control using muManager software. J Biol Methods 1 (2014).

What is claimed is:

1. A method of pharmacologically treating a subject being an MSMDS patient or a subject having or suspected of having a substitution in an actin alpha (ACTA2) locus, comprising administering a therapeutically effective amount of Sapropterin to a subject, contingent upon a presence of a genetic substitution in the actin alpha (ACTA2) locus of the subject, wherein the genetic substitution results in an amino acid change, at position 179 of SEQ 1, of arginine for histidine (R179H), leucine (R179L), cysteine (R179C), glycine (R179G) or serine (R179S).

2. The method of claim 1, further comprising: obtaining a biological sample from the subject and detecting in a biological sample, a presence of the substitution in an actin alpha (ACTA2) locus at position 179.

3. The method of claim 2, wherein the detecting in a biological sample comprises a process selected from a: primer extension assay; nucleotide incorporation assay; oligonucleotide hybridization assay; 5′ nuclease assay; an assay employing molecular beacons; DNA sequencing, genetic bit analysis (GBA) and an oligonucleotide ligation assay.

4. The method of claim 1, wherein the genetic substitution in an actin alpha 2 (ACTA2) locus results in an amino acid change, at position 179, of arginine for histidine (R179H).

5. The method of claim 1, wherein the subject is a fetus, a child, or an adult.

6. The method of claim 5, wherein the subject is less than one-year old.

7. The method of claim 2, wherein the subject is a fetus.

8. The method of claim 7, wherein the administering of a therapeutically effective amount of sapropterin is by administering to a pregnant adult thereby exposing a developing fetus.

9. The method of claim 1, wherein the administering of a therapeutically effective amount of sapropterin is by intravenous, intra-amniotic, intratracheal or intranasal route.

10. The method of claim 1, wherein the effective amount of Sapropterin is an amount sufficient to cause, in a subject, subsequent to treatment, any one or more of the following: inhibition, to some extent, of disease progression, slowing down and complete arrest; relief, to some extent, of one or more symptoms associated with the disorder; increase in a length of progression of disease following treatment; decreased mortality at a given point of time following treatment; and/or an increase in functional F-actin, an increase in the ratio between F-actin to G-actin, or a decrease in functional G-actin.

11. The method of claim 1, wherein administering a therapeutically effective amount of sapropterin is separately, sequentially, or simultaneously with an anti-platelet, anticoagulants, anti-epileptic, or inhaled active.

12. A kit comprising components for detecting in a biological sample obtained from a subject, a presence of a substitution in an actin alpha 2 (ACTA2) gene wherein the substitution in an actin alpha (ACTA2) locus results in an amino acid change in one or more of the following: at position 179, a substitution of arginine for histidine (R179H), leucine (R179L), cysteine (R179C), glycine (R179G) or serine (R179S).

13. The kit of claim 12, wherein the substitution in an actin alpha 2 (ACTA2) locus is a substitution of arginine for histidine (R179H), at position 179.
